(19) United States
(12) Patent Application Publication
(10) Pub. No.: US 2018/0163269 A1
(43) Pub. Date: Jun. 14, 2018

(54) Method for Assessing the Quality of Various Cells Including Induced Pluripotent Stem Cells

(71) Applicant: Ecole Polytechnique Federale de Lausanne, Lausanne (CH)

(72) Inventors: Didier Trono, Vufflens-Chateau (CH); Marc Friedli, Lausanne (CH); Carmen Unzu Ezquerro, Geneve (CH)

(73) Assignee: Ecole Polytechnique Federale de Lausanne, Lausanne (CH)

(21) Appl. No.: 15/578,039

(22) PCT Filed: May 27, 2016
(86) PCT No.: PCT/EP2016/061986

§ 371 Date: Nov. 29, 2017

(30) Foreign Application Priority Data
May 29, 2015 (EP) 15169880.0

(51) Int. Cl. C12Q 1/6881

(52) U.S. Cl. CPC C12Q 1/6881; C12Q 2600/158

(57) ABSTRACT

The present invention relates to a method for assessing the quality, utility and applicability of a cell according to the said method comprising the following steps: a) analyzing the expression of the TEs of said cell in order to set up an expression profile of the TEs of said cell; b) comparing the expression profile obtained in step a) to a reference.

### FIELD OF THE INVENTION

The present invention is directed to methods for assessing the quality of various cells including induced pluripotent stem cells and differentiated cells.

### BACKGROUND OF THE INVENTION

The recent discovery of factors that reprogram somatic cells from patients into induced pluripotent stem cells (iPSCs) has led to a further increase in the number of pluripotent cell lines available to the research community. Because of this progress and because iPS cells are similar to embryonic stem (ES) cells without being hampered by the same ethical and immunological concerns, it is now believed that iPSCs cells, by allowing a patient's own cells to become a source of therapeutic tissue, have the potential to become a platform for personalized medicine. Thus, iPSC has become a strong and highly promising therapeutic strategy for treating incurable human diseases.

However, a major limitation encountered with the use of such cells is that their genomic integrity may be compromised during their generation or during ex vivo culture. Indeed, at the end of the reprogramming of human somatic cells into iPS cells, several colonies that at first sight look like acceptable iPSCs are obtained, but they are not all equivalent as they have not reached the same level of reprogramming and not all of them have acquired the same differentiation potential. Moreover, some could display phenotypes incompatible with clinical applications, such as block in some differentiation pathways, predisposition to oncogenic changes, release of bioactive molecules, and altered immunogenicity.

Thus, it has become clear that not all human pluripotent cell lines are equally suited for every purpose. This suggests that any new research project should perform a deliberate and informed selection of the cell lines that are most qualified for an application of interest. However, there is little information or guidance concerning how to select cell lines that are most appropriate for each application.

There is thus an unfulfilled need for a novel, effective and efficient method that can assess the quality and developmental stage or state of a given cell, in particular induced pluripotent human cell lines, and predict their behavior.

### SUMMARY OF THE INVENTION

The inventors met the burden to show, for the first time, that the expression of transposable elements (TEs) may constitute a signature for a given cell, such as induced pluripotent stem cells as well as differentiated cells.

Thus, in a first aspect, the invention relates to a method for assessing the quality and/or utility of a cell, said method comprising the following steps:
a) analyzing the expression of the transposable elements of the said cell in order to set up an expression profile of the TEs of said cell;
b) comparing the expression profile obtained in step a) to a reference; wherein said cell is selected from the group consisting of:
   induced pluripotent stem cells (iPSC);
   pluripotent stem cells and precursor cells selected from the group consisting of embryonic stem (ES) cells, somatic stem cells, hematopoietic stem cells, leukemic stem cells, skin stem cells, intestinal stem cells, gonadal stem cells, brain stem cells, muscle stem cells, mammary stem cells, neural stem cells;
   differentiated cells; and
   cells of the embryonic developmental stages from zygote to fetus, including zygote, cells of morula, cells of blastula, cells of gastrula, cells of blastocytes.

In a first embodiment, said cell is an induced pluripotent stem cell (iPSC).

In a second embodiment, said cell is a CD34+ hematopoietic stem cell.

In a third embodiment, said cell is a differentiated cell.

In a second aspect, the invention pertains to the signatures of TEs as independently defined in table 1, table 2, table 3, table 4, table 5 and table 6. The invention also relates to the uses of said signatures for assessing the quality of a given cell.

In a first embodiment, the signatures as defined in table 1 or table 2 are used for discriminating iPSC based on their TE expression signature, that is, a TE expression pattern closest to that seen in bona fide ES cells, rather than that associated with differentiated tissue or disease phenotypes.

In a second embodiment, the signature as defined in table 3 is used for selecting hematopoietic stem cells that can be used for all relevant clinical or research applications such as hematopoietic stem cells transplantation.

In a third embodiment, the signature as defined in table 4 is used for selecting hepatocytes that are most appropriate for research or clinical applications such as disease modeling, toxicology studies or cell therapy.

In a fourth embodiment, the signature as defined in tables 5 and 6 are used for selecting respectively resting and activated CD4+ T lymphocytes.

In a third aspect, the invention relates to a kit comprising means for detecting the expression of TEs as independently defined in table 1, table 2, table 3 table 4, table 5 and table 6.

### DETAILED DESCRIPTION OF THE INVENTION

Definition

“Transposable elements” or “TEs” refer to mobile genetic elements of a genome that have or had (many have lost this ability and are now domesticated by the host cell for regulatory purposes) the ability to move from one location to another in the genome. Transposable elements constitute a normal and ubiquitous component of genomes. Transposable elements may cause genetic changes and make important contributions to the evolution of genomes by inserting into genes; inserting into regulatory sequences; or modification of gene expression through a variety of mechanisms, both transcriptional and post-transcriptional. TEs account for over half of the mouse or human genome, and their potential as insertional mutagens and transcriptional perturbators is suppressed by early embryonic epigenetic silencing. Recent findings demonstrate that certain classes of TEs or specific TE loci are essential to reach and maintain the pluripotent state.

TEs replicate in two main ways, involving either a DNA or an RNA intermediate, and they have been accordingly divided into two classes, namely: DNA transposons and retrotransposons. TEs have the ability to move from place to place in the genome, hence their designation as mobile DNAs. DNA transposons spread by a non-amplifying “cut-and-paste” mechanism, whereas retrotransposons use a copy-and-paste process that increases their genomic representation. Thus, transposable elements facilitate genome evolution, support genome structure and provide numerous cis-acting elements such as promoters, enhancers, alternative exons, transcription terminators and splice junctions to protein-coding loci or to non coding RNAs.

‘Retrotransposons”, “endogenous retroelements” and “retroposons” refer to a subset of transposable elements that go through an RNA intermediate that is reverse transcribed before re-integration at a novel genome site, in a “copy and paste” mechanism. Hence, these elements do not excise when they transpose. Instead, they make a copy that inserts elsewhere. Although transcription of retrotransposons is an integral part of their life cycle, elements may be transcriptionally active without this resulting in proliferation of the elements within the host genome.

They can be further classified based on their structure.

LTR elements or endogenous retroviruses (ERVs) are characterised by long terminal repeats and include (or included as older elements often lose part of this structure through mutation or recombination) the gag, pol, and env genes. These evolved as a consequence of retroviral infection of germ cells, so that they are inherited through generations.

Non-LTR retrotransposons include long and short interspersed repeat elements (LINEs and SINEs) and SVA elements that are a composite of sequences derived from other repeats (SINE, VNTR [variable number tandem repeat], and Alu).

There are only three highly active elements in the human genome:
(1) a subset of LINEs;
(2) Alu elements that are a family of primate-specific SINEs; and
(3) SVA elements.

Impacts of the retrotransposons on the transcriptome may be considered transcriptional (or co-transcriptional) and posttranscriptional. The former mechanisms refer to any TE related transcriptional effect and include provision of an alternative promoter that may be tissue- or stage-specific in its activity; promotion of alternative splicing either through prevention of the splicing machinery from recognising a splice acceptor site in an endogenous exon (exon skipping) or through incorporation of the TE into the mature transcript (exonization); promotion of alternative polyadenylation (poly(A)) either by providing an alternative polyadenylation signal or by promoter activity interfering with host gene transcription and causing upstream polyadenylation; and by introducing transcription factor binding sites that may confer tissue- or stage-specific expression, or link a gene into a transcriptional network.

“Embryonic stem cells” are pluripotent stem cells derived from the inner cell mass of a blastocyst, an early-stage embryo. They are unique in the ability to maintain pluripotency over significant periods in culture, making them leading candidates for use in cell therapy. Embryonic stem cell differentiation involves epigenetic mechanisms to control lineage-specific gene expression patterns. ES cell-based therapies hold great promise for the treatment of many currently intractable heritable, traumatic, and degenerative disorders. The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions.

It has become clear that pluripotency encompasses more than one stage of development. The earlier “naïve” stage is closest to the preimplantation inner cell mass or even slightly earlier stages, whereas, the “primed” stage is akin to the post-implantation epiblast. The primed ES stage is generally referred to as human ES cells (hESCs). Therefore, naïve and primed ES cells basically differ by their stage of development. Naïve ES are the developmentally earliest state described for human established cultured cells to date. Existing human ES cell lines in the later primed state can be reverse toggled to the naïve state by exposure to appropriate culture conditions.

The term “reprogramming” as used herein refers to a process that alters or reverses the differentiation state of a differentiated cell.

The term “induced pluripotent stem cell” or “iPS cell” or “iPSC” refers to a type of pluripotent stem cell artificially derived from a non-pluripotent, adult somatic cell, by compulsory dedifferentiation (reprogramming). Typically, said iPS cells are reprogrammed by the expression of the transcription factors Oct4 (also known as POU5F1), Sox2, Klf4, and c-Myc (OKSM, or other variations of reprogramming cocktails).

The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, an immuno-deficient mouse teratoma formation assay. A pluripotent cell is an undifferentiated cell.

The term “differentiated cell” refers to any primary cell that is not, in its native form, pluripotent as that term is defined herein. It is noteworthy that placing many primary cells in culture can lead to some loss of fully differentiated characteristics. Thus, simply culturing such cells is included in the term “differentiated cells” and does not render these cells non-differentiated cells (e.g. undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of self-renewal (extended passaging) without loss of growth potential, relative to primary parental cells, which generally have capacity for only a limited number of divisions in culture. In some embodiments, the term “differentiated cell” also refers to a cell of a more specialized cell type derived from a cell of a less specialized cell type (e.g. from an undifferentiated cell or a reprogrammed cell) where the cell has undergone a cellular differentiation process.

As used herein, the term “somatic cell” refers to any cell other than an ES, iPS or germ cell. In mammals, germline cells (also known as “gametes”) are the spermatozoa and oocytes, which fuse during fertilization to produce a cell called a zygote, from which the entire mammalian embryo develops.

As used herein, the term “adult cell” refers to a cell found throughout the body after embryonic development.

The term “cell line” refers to a population of largely or substantially identical cells that has typically been derived from a single ancestor cell or from a defined and/or substantially identical population of ancestor cells.

### Method for Assessing the Quality of a Cell

The inventors have showed that endogenous retroelements (EREs) are markedly upregulated during the reprogramming into iPS cells of either mouse embryonic fibroblasts, human CD34+ cells or human primary hepatocytes, with hundreds of ERE loci not associated with pluripotency aberrantly unleashed. The inventors have thus thoroughly analyzed all TE integrants (loci) during iPS reprogramming and they identified clusters of TEs that are specific of reprogramming time-points, which likely reflect cellular sub-states on the path to pluripotency.

Surprisingly, they also observe that thousands of TEs, which were broadly thought to be inactivated at this stage, are in fact highly expressed in somatic cells ranging from primary hepatocytes to neural committed cells and CD34+ hematopoietic stem cells. Therefore the inventors met the burden to show, for the first time, that the expression of retroelements may constitute a signature for a given cell such as induced pluripotent stem cells, as well as differentiated cells.

Moreover, a large fraction of expressed TEs in each cell are highly specific of that cell type, thereby providing the opportunity to accurately and predictively describe the unique expression profile of all the TEs, also referred as “transposcriptome”, of distinct cellular states. Therefore, the inventors met the burden to identify signatures that may be used as a precise marker of cellular states with broad applications in quality control.

Such a precise cell stage gauge and quality control method should prove crucial in the assessment of induced pluripotent stem cell attributes. Indeed, iPS clones are very heterogeneous in terms of phenotypes that include differentiation capacity and potentially predisposition to oncogenic changes. The inventors observed that certain iPS clones failed to differentiate in certain lineages and that this phenotype correlated with a distinct pattern of TE expression. Similarly, Yamanaka and al observed (Koyanagi-Aoi et al., PNAS 2013) that seven iPS clones that were defective for neural differentiation showed increased expression of several genes, many of which were driven by the promoter (LTR7) of a human specific endogenous retroelements (HERVH). Moreover, Ohnuki et al. (Ohnuki, PNAS 2014) proposed that general HERVH levels need to be reduced to an ES-like level in iPS clones following a peak of expression during reprogramming in order to be differentiation competent. These data are in agreement with the inventors' own demonstration of TE dysregulation during reprogramming (Friedli et al., Genome Res 2014) and recent observation that differentiation-defective iPS clones show a distinct TE expression signature.

The inventors met the burden to identify and disclose the expression profiles of the TEs of several cell stages for each of about 4 million TE integrants. The result is a highly accurate and specific signature akin to a unique barcode descriptive and predictive of each cell stage or state. These TE signatures are approximately two orders of magnitude above that of tissue-specific genes, with thousands of TEs uniquely expressed in a specific cell type.

Therefore, in a first aspect, the present invention relates to a method for assessing the quality and/or utility of a cell, said method comprising the following steps:
a) analyzing the expression of the TEs of said cell in order to set up an expression profile of all TEs, more precisely of all TE integrants (loci) of said cell;
b) comparing the expression profile obtained in step a) to a reference;
   wherein said cell is selected from the group consisting of:
induced pluripotent stem cells (iPSC);
pluripotent stem cells and precursor cells selected from the group consisting of embryonic stem (ES) cells, somatic stem cells, hematopoietic stem cells, leukemic stem cells, skin stem cells, intestinal stem cells, gonadal stem cells, brain stem cells, muscle stem cells, mammary stem cells, and neural stem cells;
differentiated cells; and
cells of the embryonic developmental stages from zygote to fetus, including zygote, cells of morula, cells of blastula, cells of gastrula, cells of blastocytes.

The method of the invention allows for a high throughput screening which provides for the rapid identification and selection of cells, which are suitable for further uses such as research, modeling, therapeutic or bioprocessing applications. The invention thus harnesses the power of genetic by providing specific signatures of TE expression that give essential information regarding a cell line.

Step a) of the method of the invention consists of an analysis of the expression of all TE integrants (loci) of a cell. Step a) allows establishing the expression profile of the TE of said cell. Preferably, said TEs are retrotransposons.

As used herein, the expression “expression profile of the TEs” refers to qualitative and/or quantitative expression of the TEs in a cell. The expression profile is a repository of the expression level data that can be used to compare the expression levels of different TEs, in whatever units are chosen. Put in other words, step a) consists in setting up a collection of all the TEs of the cells, i.e. setting up the “transposcriptome” of said cell. In the context of the invention, the terms “expression profile of the TEs”, “transposable element (TE) transcriptome” and “transposcriptome” can be used interchangeably, and refer to the collection of all transcripts containing TE and TE-derived sequences in a specific cell. Basically, the transposcriptome encompasses all the transposable elements transcribed in any cell type or developmental stage/state.

In a preferred embodiment, the transcriptome according to the invention refers to the collection of all transcripts containing retrotransposons and retrotransposons-derived sequences in a specific cell.

The inventors met the burden to specifically identify the loci of the TEs (TE integrants). Thus, the expression “transposcriptome” preferably refers to the collection of all transcripts containing TE and TE-derived sequences in a specific cell, and the precise identifications of the locus from which they originate.

Typically, step a) of analyzing the expression profile of TEs consists in identifying and quantifying the transcripts originating from all TEs, more precisely all TE integrants (loci).

Said step is carried out by RNA Sequencing, also referred to as “RNAseq”. RNAseq has been widely adopted as a gene-expression measurement tool due to the detail, resolution, and sensitivity of transcript characterization that the technique provides. It falls within the general knowledge of person skilled in the art to carry out a RNA sequencing for identifying accurately mapping, and quantifying TEs.

In another embodiment, the step of analyzing the expression of TEs of a cell consists in identifying the transposable element integrants within the DNA of the cell. Said step is carried by the unambiguous mapping of individual TE reads (sequences) to specific unique genomic loci. This step requires:
Sufficient depth of sequencing (typically >40 million reads per RNA sample);
Sufficient length of reads (100 bp or more);
Paired-end and stranded protocols desirable.

Typically, step a) can be performed on an Illumina platform according to the manufacturer's standard protocols.

As used herein, “cell quality” relates to technical features of a cell that render such cell appropriate for given intended uses. Typically, quality control relates to features such as cell identity/morphology, cell phenotype, cell genotype, levels of cell contamination, degree of pluripotency, differentiation or reprogramming potential, cell viability, contamination levels, and/or cell safety especially with regards to potential predisposition to oncogenic changes or uncontrolled and anarchic cell growth.

For stem cells, quality is usually defined as self-renewal capacity, expression of specific markers, differentiation ability, ability to be converted to the naïve state, and absence of detrimental phenotypes such as predisposition to oncogenic changes, release of bioactive molecules, and altered immunogenicity.

Preferably, as used herein, a cell which proves to be of acceptable quality is a cell that does not show any potential predisposition to oncogenic changes or uncontrolled and anarchic growth. Thus, in the context of the present invention, the expression “assessing the quality of a cell” preferably refers to the determination of an absence of a potential predisposition to oncogenic changes or uncontrolled and anarchic cell growth.

As used herein, the expression “cell utility” or “cell use” refers to the intended purposes of the target cell. Said purposes include but are not limited to research, therapeutic or diagnostic purposes.

In prior art, the assessment of the quality of a cell is implemented with tests that may involve the use of DNA profiling techniques, immunohistochemistry, alkaline phosphatase staining, flow cytometry, gene expression analysis (perhaps using expression arrays and the like), blood group typing, karyotypes, microorganism screening (using PCR and immunological based techniques), teratoma and embryoid body formation (particularly relevant where the pluripotency of a stem cell is being tested) and simple live/dead (trypan blue) stains to determine viability. By establishing a reference indicative of a certain cell “quality standard”, the inventors developed a method that allows controlling the quality of cells by comparison of TE transcriptome (transposcriptome) to a reference.

It should be understood that the term “standard” or “quality standard” may relate to defined criteria or features which any given cell must exhibit prior to being used. Such standards may be set by regulatory bodies but may also relate to locally determined cell features and/or characteristics which render cells suitable for particular uses—for example uses in assays, or in therapeutic purposes.

Typically, the reference of step b) can be determined experimentally, empirically, or theoretically. Preferably, said reference is the expression profile of the TEs of a cell having known or defined qualities. More preferably, said reference is a signature of TE expression of a cell having known or defined qualities.

In a first embodiment, said cell is an induced pluripotent stem cell.

The method of the invention thus relates to a method of characterization of various cells, especially pluripotent stem cells such as induced pluripotent stem cells (iPSCs). iPSC are very heterogeneous in terms of phenotypes. Presently, existing methods cannot predict how a pluripotent stem cell line will behave in a given directed differentiation paradigm.

The method of the invention addresses the limitations of prior art by enabling to forecast the differentiation efficiency of iPSC and their quality, especially with regards to the absence of any potential predisposition to oncogenic changes or uncontrolled and anarchic cell growth. The method of the invention thus allows a quality control that proves crucial in the assessment of iPSC.

Preferably, step b) consists in a step b1) of comparing the expression profile of step a) to the expression profile of the TEs of a naïve embryonic stem cell or a primed embryonic stem cell, preferably of a primed embryonic stem cell. In this embodiment, said iPSC is considered as showing an acceptable quality and/or to show genomic integrity if the expression profile obtained in step a) comprises at least 80%, 85%, 90%, 95% or more preferably 100% of the TEs of the expression profile of said naïve embryonic stem cell or said primed embryonic stem cell.

Alternatively, said step b) consists in a step b2) of comparing the expression profile obtained in step a) to the expression of all the TEs disclosed in table 1 or table 2. In this embodiment, the iPSC is considered as showing an acceptable quality and/or to show genomic integrity if at least 80%, preferably 85%, preferably 90%, preferably 95% or more preferably 100% of the TEs as disclosed in table 1 or 2 are present in the expression profile as obtained in step a).

As used herein, the expression “genomic integrity” refers to an apparent absence of mutation or any risk of malignant transformation of the iPSC's genome.

Preferably, said step b) consists in a step b2) of comparing the expression profile obtained in step a) to the expression of all the TEs disclosed in table 2.

Table 1 represents the TEs expressed in a naïve embryonic stem cell. The list of TEs of table 1 thus constitutes a unique signature of naïve embryonic stem cells.

Table 2 represents the TEs expressed in a primed embryonic stem cell. The list of TEs of table 1 thus constitutes a unique signature of primed embryonic stem cells.

As used herein, the expressions “signature”, “signature of TE expression” or “TE signature” refer to the expression of TEs in a given cell that proved to be specific to a given cell type, a stage of development and/or a state of said cell. It is noteworthy that a “signature” in the sense of the present invention does not comprise all the TEs of the cells (i.e. the transposcriptome). Quite the reverse, a signature comprises only the TEs that are found to be specific to a given cell type, state or stage of development.

By comparing the TE transcriptome of an iPS cell to the TE signatures of naïve ES or primed ES cells, as respectively defined in tables 1 and 2, the person skilled in the art can identify an iPS cell that shows high quality, especially with regards to its differentiation ability.

Thus, in a specific embodiment, the invention relates to a method for assessing the quality of an iPS cell comprising the steps of:
a) analyzing the expression of the TEs of said iPS cell in order to set up an expression profile of the TEs of said iPS cell; and
b) comparing the expression profile obtained in step a) to the expression of all the TEs disclosed in table 1 or 2, preferably in table 2.

In a second embodiment, said cell is a hematopoietic stem cell.

In this embodiment, step b) consists in a step b3) of comparing the expression profile obtained in step a) to the expression of all the TEs disclosed in table 3. In this embodiment, the hematopoietic stem cell is considered as showing an acceptable quality if at least 80%, preferably 85%, preferably 90%, preferably 95% or more preferably 100% of the TEs disclosed in table 3 are present in the expression profile as obtained in step a).

Table 3 represents the transposable elements expressed in a CD34+ hematopoietic stem cell. The list of the transposable elements of table 3 thus constitutes a unique signature of CD34+ hematopoietic stem cells.

Thus, in a specific embodiment, the invention relates to a method for assessing the quality of a hematopoietic stem cell comprising the steps of:
a) analyzing the expression of the TEs of said hematopoietic stem cell in order to set up an expression profile of the TEs of said cell; and
b) comparing the expression profile obtained in step a) to the expression of all the TE disclosed in table 3.

In this particular embodiment, the method of the invention allows the identification of hematopoietic stem cells that are of higher quality for broad applications. Indeed abnormal CD34+ cell levels are known to be associated with a poor prognosis of several cancers, especially acute myeloid leukemia. Acute myeloid leukemia (AML) is a malignancy of immature cells. Intensive chemotherapy is the mainstay of treatment, but primary resistance and relapse after an apparent initial remission are frequently observed. Various factors are considered to be responsible for the difference in response to chemotherapy and to serve as prognostic parameters predicting outcome of therapy.

One of the promising therapeutic strategies for said disease may require transplantation of relevant CD34+ hematopoietic cells. The method of the invention allows the identification hematopoietic cells that are highly appropriate for such transplantation. The method of the invention thus allows the identification of hematopoietic stem cells that constitute good candidates for hematopoietic stem cell transplantation.

In a third embodiment, said cell is a differentiated cell.

Preferably, said cell is selected among neuronal cells and hepatocytes. More preferably, said cell is a hepatocyte.

The inventors have met the burden to show, for the very first time, that differentiated cell line can be identified and characterised thanks to the expression of TEs. They thus have shown that the method of the invention may give sensitive and important information regarding differentiated cells. Said information include: the cell type, the cell quality, and the degree of differentiation of said cell, the activation state of said cell. This information is critical for the assessment of cells envisioned for each application whether for basic research, diseases, modelling, drug screening or clinical applications.

In this embodiment, step b) consists in a step b4) of comparing the expression profile obtained in step a) to the expression of all the TEs as disclosed in table 4. In this embodiment, the hepatocyte is considered as showing an acceptable quality if at least 80%, preferably 85%, preferably 90%, preferably 95%, or more preferably 100% of the transposable elements disclosed in table 4 are present in the expression profile as obtained in step a).

Thus, in a specific embodiment, the invention relates to a method for assessing the quality of a hepatocyte comprising the steps of:
a) analyzing the expression of the TEs of said hepatocyte in order to set up an expression profile of the TEs of said cell; and
b) comparing the expression profile obtained in step a) to the expression of all the TEs disclosed in table 4.

In a fourth embodiment, said cell is a CD4+ T lymphocyte.

The inventors have shown that CD4+ T lymphocytes can be identified and characterised thanks to the expression of TEs. The method of the invention allows the identification of resting and activated CD4+ T lymphocytes.

In this embodiment, step b) consists in a step b5) of comparing the expression profile obtained in step a) to the expression of all the TEs as disclosed in tables 5 and 6.

The CD4+ T lymphocyte is considered as resting if at least 80%, preferably 85%, preferably 90%, preferably 95%, or more preferably 100% of the transposable elements disclosed in table 5 are present in the expression profile as obtained in step a).

The CD4+ T lymphocyte is considered as activated if at least 80%, preferably 85%, preferably 90%, preferably 95%, or more preferably 100% of the transposable elements disclosed in table 6 are present in the expression profile as obtained in step a).

### Signature and Uses Thereof

In a second aspect, the invention relates to specific signatures of TE expression. As indicated above, said signatures give various indication regarding a given cell line. The invention thus relates to uses for assessing the quality of a cell line.

Said signature gives comprehensive information regarding the quality of a cell and allow the discriminating cells depending on their quality, especially when compared to the expression profile of TEs of a cell having known or defined qualities.

The use of signature may allow discriminating induced pluripotent cells depending on their quality, especially their differentiation ability, developmental stage/state, and safety. The “differentiation ability” of an iPSC refers to its ability of differentiating in any kind of cells.

The use of said signature may also discriminate differentiated cells depending on their predisposition to oncogenic changes or uncontrolled and anarchic growth.

The invention thus pertains to the signatures of TEs as independently defined in table 1, table 2, table 3 and table 4.

The inventors have shown that said signatures are highly useful for assessing the quality of a given cell.

More precisely, the signatures as defined in tables 1 and 2 are used for assessing the quality of an iPSC. More specifically, the signature disclosed in tables 1 and 2 are compared to the expression profile of the TEs of a given iPSC for determining the general quality and differentiation potential of said iPSC. Thus, in a first embodiment, said signature is useful for discriminating cells depending on their differentiation ability. Said signature is also useful for discriminating cells depending on their ability to convert to the naïve state, their safety, and their general research or clinical applications.

In a second embodiment, the signature as defined in table 3 is useful for assessing the quality of hematopoietic stem cells. More specifically, said signature is useful for selecting hematopoietic stem cells that are appropriate for research or clinical applications including hematopoietic stem cells transplantation.

In a third embodiment, the signature as defined in table 4 is useful for assessing the quality of a hepatocyte. More specifically, said signature is useful for selecting hepatocytes that do not show a predisposition to oncogenic changes or uncontrolled and anarchic growth and that are generally appropriate for research, disease modeling, drug screening, and cell therapy.

In a fourth embodiment, the signatures as defined in tables 5 and 6 are used for selecting respectively resting and activated CD4+ T lymphocytes.

### Kits

The inventors have carried out a comprehensive analysis of the transposable elements of different kinds of cells.

Thus, in a third aspect, the invention relates to a kit comprising means for detecting the expression of the transposable elements as independently defined in tables 1, 2, 3, 4, 5 and 6.

TABLES

Lengthy table referenced here
US20180163269A1-20180614-T00001
Please refer to the end of the specification for access instructions.

Lengthy table referenced here
US20180163269A1-20180614-T00002
Please refer to the end of the specification for access instructions.

Lengthy table referenced here
US20180163269A1-20180614-T00003
Please refer to the end of the specification for access instructions.

Lengthy table referenced here
US20180163269A1-20180614-T00004
Please refer to the end of the specification for access instructions.

Lengthy table referenced here
US20180163269A1-20180614-T00005
Please refer to the end of the specification for access instructions.

Lengthy table referenced here
US20180163269A1-20180614-T00006
Please refer to the end of the specification for access instructions.

The invention will be further illustrated by the following examples. However, these examples should not be interpreted in any way as limiting the scope of the present invention.

### EXAMPLE

Through an ongoing study of the RNAseq data using state of the art transpostcriptome analysis pipeline, the inventors have identified thousands of TE loci specific and likely predictive of cellular states.

When analyzing the transpostcriptome of intermediate reprogramming time-points of CD34+ hematopoietic on the path to induced pluripotency (iPSCs), the inventors rapidly observed clusters of TEs scattered throughout the genome with acute expression patterns peaking at specific days of reprogramming. For example, the LTRs associated with HERVH-int (a human restricted ERV) were transcribed at precise time-points likely representing intermediate cellular or developmental stages, as iPSC reprogramming represents a paradigm of reversed developmental course. Of 50 full-length HERVH integrants expressed at day 4 of reprogramming, 39 (78%) were associated with LTR7B while LTR7C vastly predominates at other time-points (e.g. 31/32, 97% at d14).

The inventors hypothesized that many more integrants specific of intermediate cell stages could be observed and eventually used as markers of cell state and quality. To this end, they generated and analyzed RNAseq of several somatic cell types including primary human hepatocytes, CD34+ hematopoietic stem cells, neural committed cells, along with iPSC, differentiation intermediates and human ES cells as comparisons. As predicted, the inventors found thousands of specific TE loci uniquely expressed in each somatic cell type thus providing an accurate and high-resolution description cell states. For example, at least 2′500 TE loci where only expressed in CD34+ cells as compared to hepatocytes, neural committed cells, ES, iPSC, and differentiation intermediates on the path to hepatocyte (endoderm) or neural commitment (ectoderm). Likewise, thousands of TEs uniquely tagged primary human hepatocytes and neural committed cells.

The inventors next compared human naïve ES cells and their primed counterparts. Again, they found thousands of TEs specifically tagging each of these pluripotent states.

When looking at overrepresented TEs, they found that SVA elements, particularly SVA-D, where highly enriched in the catalogue of naïve ES specific TEs. Indeed, of 892 over-expressed SVA-D in one state compared to the other (2 fold cut-off, p val<0.05), 891 (99.9%) were more expressed in naïve ES cells. Moreover of the top 10 over-expressed TEs in naïve versus primed ES cells, 6 were SVA-D integrants and 1 was a SVA-F, with expression fold changes higher than 1000 fold. Interestingly, all SVA subfamilies (SVA-F 419/422, SVA-E 155/165, SVA-C 167/168, SVA-B 169/177) bar SVA-A were over-represented in naïve ES cells. Most SVA-As were unchanged (143/226) when comparing the two states. As previously reported, HERVH and LTR7 were the two families most overrepresented in the catalogue of primed ES cells. Interestingly, the potential controllers such as KRAB-ZFPs, are also highly specific of cellular sub-states. This indicates that they indeed rewire species-specific transcriptional networks through the specific binding of species-restricted TEs.

The postranscriptome of hepatocytes and CD34+ hematopoietic stem cells does not show such clear cut enrichment for specific TE families. However, the inventors provide a list of hundreds of TEs providing specific signatures for both cell types. CD34+ hematopoietic stem cells seem to display a modest enrichment for expression of LTR retrotransposons, while hepatocytes show a mild bias toward LINES.

### RNA-seq Method for the Quantification of Integrant Specific TE Expression.

In order to identify TEs specific and predictive of cellular stages and quality, the inventors have generated the following RNAseq datasets (100 bp, single end, non stranded unless otherwise stated):
Reprogramming CD34+ to iPSC: 9 intermediate time-points of RNAseq samples (same donor), over 21 days, 6 resulting iPS clones;
Somatic cells, differentiation, and reprogramming: 3 CD34+ (same donor) samples, 1 iPSC (unrelated to above) d1, d3, d4 of its neural differentiation, 1 resulting neural committed cell line, 3 primary hepatocytes, 3 reprogramming time-points of hepatocytes (hep) on the path to pluripotency, 2 resulting iPS clones, 14 differentiation time points of hep iPSC on the path to hepatocytic differentiation (3 different starting clones, 2 Hep-like cells (final differentiation stage);
Human ES cells: 11 RNA Seqs: 3 RNA-seqs from H1 Trono lab, 6 UCLA (1-6), H1 from UCLA, H9 UCLA; and
Human primed and Naïve ES cells: 12 RNA-seqs, 100 bp, paired-end, stranded. 8 naïve (3 of which embryo-derived), 4 primed.

Conclusion

The inventors have showed that the specificity of each cell type can be accurately described and predicted using the transpostcriptome with tens of thousands expressed in each cell type providing a unique signature to each cell state, far more accurate than the expression of tissue specific genes.

The results confirm that the method presented here can be used for a broad range of cell stage and quality assessment such as quality control of various types of cell, such as iPSC. Good iPS clones should display a TE signature closely resembling that of human ES cells and should be convertible to the naïve state of pluripotency, with the matching TE expression pattern.

LENGTHY TABLES
The patent application contains a lengthy table section. A copy of the table is available in electronic form from the USPTO web site (http://seqdata.uspto.gov/?pageRequest=docDetail&DocID=US20180163269A1). An electronic copy of the table will also be available from the USPTO upon request and payment of the fee set forth in 37 CFR 1.19(b)(3).

1. A method for assessing the quality and/or utility of a cell, said method comprising the following steps:
(a) analyzing the expression of the transposable elements (TEs) of said cell in order to set up an expression profile of the TEs of said cell;
(b) comparing the expression profile obtained in step (a) to a reference,
wherein said cell is selected from the group consisting of:
induced pluripotent stem cells (iPSC);
pluripotent stem cells and precursor cells selected from the group consisting of embryonic stem (ES) cells, somatic stem cells, hematopoietic stem cells, leukemic stem cells, skin stem cells, intestinal stem cells, gonadal stem cells, brain stem cells, muscle stem cells, mammary stem cells, neural stem cells;
differentiated cells; and
cells of the embryonic developmental stages from zygote to fetus, including selected from the group consisting of zygote, cells of morula, cells of blastula, cells of gastrula, cells of blastocytes.

2. The method according to claim 1, wherein the expression profile of step a) consists of a collection of all the TEs of said cell.

3. The method according to claim 1, wherein step a) is performed by RNA sequencing.

4. The method according to claim 1, wherein said cell is an induced pluripotent stem cell (iPSC).

5. The method according to claim 4, wherein:
step (b) comprises a step (b1) of comparing the expression profile obtained in step (a) to the expression profile of the TEs of a naive embryonic stem cell or a primed embryonic stem cell; and
said iPSC is considered as showing an acceptable quality and/or to show genomic integrity if the expression profile obtained in step (a) comprises at least 80% of the TEs of the expression profile of said naive embryonic stem cell or said primed embryonic stem cell.

6. The method according to claim 4, wherein:
said step (b) comprises a step (b2) of comparing the expression of TEs of step (a) to the expression of all the TEs disclosed in table 1 or table 2; and
said iPSC is considered as showing an acceptable quality and/or to show genomic integrity if at least 80% of the TEs disclosed in table 1 or 2 are present in the expression profile as obtained in step a).

7. The method according to claim 1, wherein said cell is a hematopoietic stem cell.

8. The method according to claim 7, wherein:
said step (b) comprises a step (b3) of comparing the expression profile obtained in step a) to the expression of all the TEs disclosed in table 3; and
said hematopoietic stem cell is considered as showing an acceptable quality if at least 80% of the TEs disclosed in table 3 are present in the expression profile obtained in step (a).

9. The method according to claim 1, wherein said differentiated cell is a hepatocyte.

10. The method according to claim 9, wherein:
said step (b) comprises a step (b4) of comparing the expression profile obtained in step (a) to the expression of all the TEs disclosed in table 4; and
said hepatocyte is considered as showing an acceptable quality if at least 80% of the TEs disclosed in table 4 are present in the expression profile as obtained in step (a).

11. Signatures of TE expression as independently defined in table 1, table 2, table 3, table 4, table 5 and table 6.

12-17. (canceled)

18. A kit comprising means for detecting the expression of the TEs as independently defined in tables 1, 2, 3, 4, 5 and 6.

19. The method according to claim 1, wherein:
said cell is an iPSC and said TEs are those defined in table 1 or table 2;
said cell is a hematopoietic stem cell and said TEs are those defined in table 3;
said cell is a differentiated cell which is a hepatocyte and said TEs are those defined in table 4; or
said cell is a differentiated cell which is a CD4+ T lymphocyte and said TEs are those defined in tables 5 or 6.
